(19) United States
(12) Patent Application Publication
(10) Pub. No.: US 2024/0337671 A1
(43) Pub. Date: Oct. 10, 2024

(54) METHOD FOR QUANTIFYING ENDOTOXINS IN A BIOLOGICAL SAMPLE

(71) Applicant: BIOMERIEUX, Marcy L'Etoile (FR)

(72) Inventors: Matthieu GOUJARD, Grenoble (FR); Patrick BROYER, Saint-Cassien (FR); Frédéric PINSTON, Grenoble (FR)

(73) Assignee: BIOMERIEUX, Marcy L'Etoile (FR)

(21) Appl. No.: 18/575,986

(22) PCT Filed: Jul. 19, 2022
(86) PCT No.: PCT/FR2022/051428

§ 371 Date: Jan. 2, 2024

(30) Foreign Application Priority Data
Jul. 19, 2021 (FR) FR2107772

(51) Int. Cl. G01N 33/92; G01N 21/75; G01N 21/76; G01N 21/77

(52) U.S. Cl. CPC G01N 33/92; G01N 21/76; G01N 2021/752; G01N 2021/755; G01N 2021/7786; G01N 2333/195

(57) ABSTRACT

A method for quantifying endotoxins in a biological sample with an analysis medium added to the field of view of an imager, the analysis medium including at least one analysis chamber and a plurality of reference chambers, including the steps of, for each of a plurality of measuring times of a measuring period: a) acquiring an image and determining light intensity valves in the reference and analysis chambers; b) determining a calibration relationship connecting the light intensity value and the concentration at this measuring time; c) determination of at least one endotoxin concentration in a biological sample from said calibration relationship and the intensity value in the analysis chamber at said measurement time; the method subsequently including the step of d) determining a temporal evolution of an endotoxin concentration in the biological sample over the course of the measuring period from the endotoxin concentration for a plurality of measurement times.

### TECHNICAL FIELD

The present invention relates to the field of biological sample analysis, and more specifically concerns a process for quantifying endotoxins in a biological sample by means of a measuring instrument.

### TECHNOLOGICAL BACKGROUND

Endotoxins are toxins located in the outer membrane of certain Gram-negative bacteria, which are lipopolysaccharidic (LPS) in nature and thermostable. They are pyrogenic substances, i.e. they can cause high fevers. Pharmacopeia standards require the absence of such substances in pharmaceutical products which come into contact with the bloodstream or the central nervous system, such as injectable medicaments or medical devices. It is also recommended that endotoxins be quantified in raw materials such as water or in-process products.

Endotoxin detection currently relies mainly on the use of reagents developed from a purified fraction of blood from horseshoe crabs, a family of crabs that are endangered in Asia and protected in the USA, whose blood has the property of coagulating in the presence of minute amounts of bacterial endotoxins. A novel approach based on horseshoe crab recombinant Factor C (rFC) proteins has been developed, making it possible to dispense with the use of horseshoe crab blood and thus to detect the presence of endotoxins.

In a typical endotoxin detection process, several endotoxin solutions at standardized concentrations are prepared, for example at 50 EU/mL, 5 EU/mL, 0.5 EU/mL. EU stands for Endotoxin Unit, and is a measure of endotoxin activity, equivalent to the International Unit (IU).

This approach still relies on quantitative in vitro determination of endotoxin in pharmaceutical, biological and environmental samples. These tests are very exacting and require many operator handling steps, including the preparation of standard dilutions and internal controls. These manual preparation steps are time-consuming and may lead to variable or even invalid results.

In addition, current processes are based solely on the results obtained on conclusion of a fixed measurement period, for example 90 minutes, during which the biological sample to be analyzed is placed at a given temperature, typically about 37° C. This fixed measurement period is common to all measurements, and has been chosen in advance so as to be long enough to allow full completion of the various reactions that are liable to take place with different dynamics. Consequently, for most measurements, this measurement period is much longer than necessary, and despite this, it may still be too short for some measurements in particular cases. In addition, in the event of a faulty measurement configuration, for example an operating error, a possible problem may only be detected at the end of the measurement period, when an attempt is made to utilize the erroneous results.

### PRESENTATION OF THE INVENTION

The invention is thus directed toward making it possible to monitor the temporal evolution of an endotoxin concentration in the biological sample over the course of the measurement period, reliably at each measurement instant.

To this end, the invention proposes a process for quantifying endotoxins in a biological sample via an analytical instrument comprising an imager defining a field of view, an analytical support being introduced into the field of view of the analytical instrument, said analytical support comprising at least one analysis chamber configured to receive said biological sample and a plurality of reference chambers configured to receive a reference liquid, the process first involving placing the biological sample in said analysis chamber and reference liquid in the plurality of reference chambers, reference chambers being provided with reference reagents and different concentrations of endotoxins, said reference reagents being capable of causing a luminescence reaction in the presence of the reference liquid as a function of the endotoxin concentration in the corresponding reference chamber, the analysis chamber being provided with analytical reagents that are capable of causing a luminescence reaction in the presence of endotoxins of the biological sample,
the process comprising, for each measurement instant of a measurement period, the acquisition at said measurement instant of an image of the analytical support and the determination, from said image of the analytical support, of an analysis chamber light intensity value for said measurement instant, the process also comprising, for a plurality of measurement instants:
   the determination, from the image of the analytical support, of light intensity values of reference chambers for said measurement instant,
   the determination, from the light intensity values of reference chambers for said measurement instant, of a calibration relationship linking the light intensity value and the endotoxin concentration at said measurement instant;
the process also comprising, for a plurality of measurement instants:
   the determination of at least one measurement of endotoxin concentration in the biological sample at said measurement instant from a calibration relationship for said measurement instant and the analysis chamber intensity value at said measurement instant; and
   the determination of a temporal evolution of the endotoxin concentration measurement in the biological sample over the measurement period from endotoxin concentration measurements for several measurement instants.

The invention is advantageously completed by the following features, taken alone or in their various possible combinations:
the process then involves performing an action as a function of the temporal evolution of the endotoxin concentration measurement;
the action taken involves stopping the process or issuing an alert as a function of the stability of said temporal evolution of the endotoxin concentration measurement or of a decrease in the endotoxin concentration measurement;
a calibration relationship is determined for each measurement instant of the measurement period;
the calibration relationship for a measurement instant is a calibration relationship determined from the light intensity values of the reference chambers of a preceding measurement instant;
the temporal evolution of a measurement of endotoxin concentration in the biological sample is determined several times during the measurement period, each time following a measurement instant taken into account in said temporal evolution;
analytical reagents present in analysis chambers and reference reagents present in reference chambers comprise a recombinant factor C and a fluorogenic substrate of the reference chambers comprising endotoxins at predetermined concentrations;
at least one reference chamber is free of endotoxin;
the analytical support comprises a plurality of analysis chambers, and the determination of an analysis chamber light intensity value for said measurement instant comprises the determination of a statistically representative value of light intensity values of a plurality of said analysis chambers.

The invention also relates to an analytical instrument comprising an imager defining a field of view, the instrument being configured to receive an analytical support in the field of view of the imager, said analytical support comprising at least one analysis chamber configured to receive a biological sample, and a plurality of reference chambers configured to receive a reference liquid, the reference chambers being provided with reference reagents and reference concentrations, the reference reagents being capable of causing a luminescence reaction in the presence of the reference liquid as a function of the endotoxin concentration in the corresponding reference chamber, the analysis chamber being provided with analytical reagents that are capable of causing a luminescence reaction in the presence of endotoxins of the biological sample, the system being configured to perform at least the steps of the process according to the invention.

The invention also relates to a system comprising an analytical instrument and an analytical support according to the invention.

### PRESENTATION OF THE FIGURES

Other features, aims and advantages of the invention will become apparent from the following description, which is purely illustrative and nonlimiting, and which should be read in conjunction with the appended drawings, in which:

FIG. 1 schematically illustrates an analytical support arranged in the field of view of an imager of the measuring instrument, according to a possible embodiment of the invention;

FIG. 2 shows an example of an analytical support including a plurality of chambers, which may be used for inserting a biological sample to be analyzed, according to a possible embodiment of the invention;

FIG. 3 is a diagram showing process steps according to a possible embodiment of the invention;

FIG. 4 schematically shows treatments applied to the image of a chamber, according to a possible embodiment;

FIG. 5 shows an example of the temporal evolution of light intensity values of reference chambers according to a possible embodiment of the invention;

FIG. 6 shows examples of straight lines representing calibration relationships linking light intensity value and concentration at that measurement instant, according to a possible embodiment of the invention;

FIG. 7 shows a curve illustrating an example of temporal evolution of an analyte concentration in the biological sample over the measurement period, according to a possible embodiment of the invention.

FIG. 8 shows a curve illustrating an example of the temporal evolution, over the measurement period, of the logarithm of an average of light intensity values of analysis chambers receiving the biological sample, according to a possible embodiment of the invention.

### DETAILED DESCRIPTION

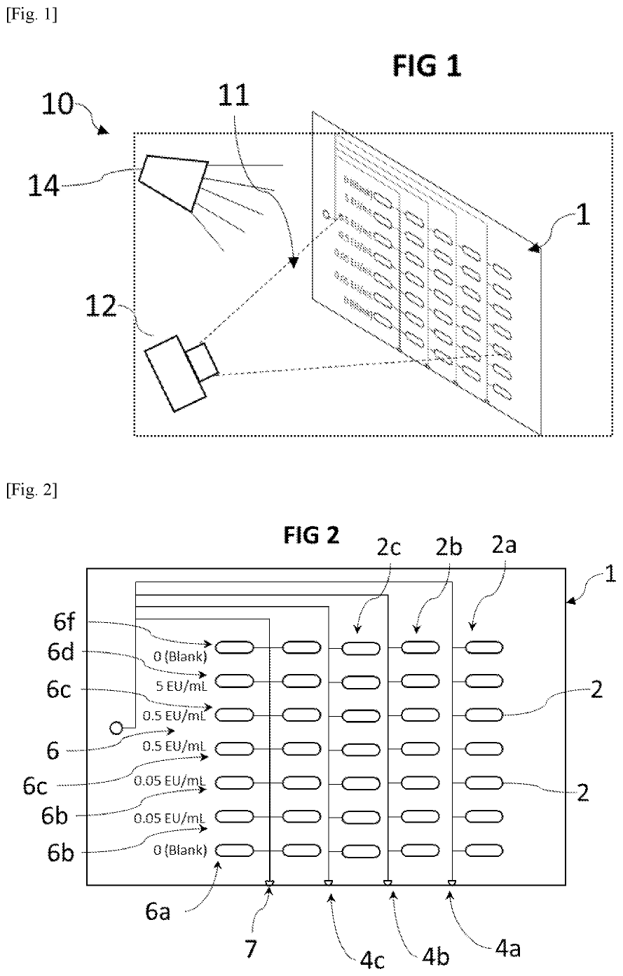

With reference to FIG. 1, the process for analyzing a biological sample is performed by means of an analytical instrument 10 comprising an imager 12, typically a fluorimeter, defining a field of view 11, and an analytical support 1 inserted into the analytical instrument 10, within the field of view 11 of the imager 12. The analytical support 1 comprises at least one analysis chamber configured to receive the biological sample, generally in liquid form, and a plurality of reference chambers configured to receive a reference liquid. The measuring instrument 10 may also include a light source 14 configured to illuminate the field of view 11 with light whose wavelength is capable of highlighting a fluorescence phenomenon, i.e. of causing the emission of fluorescent light after excitation of a fluorophore. The wavelength of the illumination light is thus chosen as a function of the fluorophore to be detected, and more particularly as a function of a fluorophore excitation wavelength. Similarly, the imager 12 may be equipped with a detection filter with an optical bandwidth corresponding to an emission wavelength of the fluorophore to be detected. Unless otherwise indicated, the light source illuminates the field of view 11 during image acquisition.

The measuring instrument may also comprise components used for data processing, such as a processor, a memory or a power supply.

FIG. 2 shows an example of an analytical support 1 including a plurality of analysis chambers 2 which may be used for installing a biological sample to be analyzed. The analysis chambers 2 are organized here in three networks corresponding to three different dilutions of the sample. In the example shown, a first network is defined by a first feed channel 4a designed to feed first analysis chambers 2a with a first dilution, for example 1:1, of the biological sample. The first network thus combines all the first analysis chambers 2a, which are distributed in one direction, here the vertical direction in FIG. 2. A second network is defined by a second feed channel 4b designed to feed second analysis chambers 2b with a second dilution, for example 1:10, of the biological sample. The second network thus combines all the second analysis chambers 2b, which are distributed in one direction, here the vertical direction in FIG. 2. A third network is defined by a third feed channel 4c designed to feed third analysis chambers 2c with a third dilution, for example 1:100, of the biological sample. The third network thus combines all the third analysis chambers 2c, which are distributed in one direction, here the vertical direction in FIG. 2. If in this example three dilutions are used, more or fewer dilutions may be used, defining as many networks of analysis chambers 2. Preferably, however, the analytical support comprises several analysis chambers 2, preferably combined in two networks so as to receive the biological sample at two different dilutions.

The analytical support 1 also comprises a plurality of reference chambers 6 configured to receive a reference liquid. Each reference chamber 6 includes reference reagents previously arranged in the chamber, and also different concentrations of endotoxins. Preferably, however, at least one reference chamber 6 is a control chamber not comprising any endotoxin. Preferably, a matrix is then present in the control chamber, with a deposit derived from a solution making it possible to obtain chemical characteristics close to those of the endotoxins. This is, for example, a polyether such as polyethylene glycol (or PEG), or an organic polymer such as polyvinylpyrrolidone, or PVP.

The reference reagents are capable of causing a luminescence reaction as a function of the endotoxin concentration in the corresponding reference chamber. The term “luminescence” means light emission without incandescence, for instance fluorescence. Typically, these reference reagents are dehydrated, and the reference chambers 6 are configured to receive a reference liquid allowing reactions to take place on contact with the reference reagents. Typically, this reference liquid is “endotoxin-free water”. Such endotoxin-free water generally meets specific requirements other than the absence of endotoxin, such as guaranteed sterility, filtering at less than 1 μm, etc. The different endotoxin concentrations in the reference chambers 6 containing same typically cover a range of concentrations with factors from 1 to several tens, or even from 1 to 100. Preferably, the endotoxin is a LipoPolySaccharide (LPS) produced only by Gram-negative bacteria, such as Escherichia coli, Salmonella enteritidis, Legionella pneumophila, Campylobacter jejuni, Vibrio cholerae, Shigella dysenteriae, Pseudomonas aeruginosa and many others.

Different reference chambers 6 are provided with at least two different endotoxin concentrations, and preferably at least three different endotoxin concentrations. The reference reagents may comprise a buffer, recombinant factor C and a fluorogenic substrate. The reagents in the reference chambers 6 comprise, for example, a detection agent in an inactive state in the absence of activation, which may comprise endotoxins, an agent for activating the detection agent comprising an enzyme and a fluorogenic substrate, and a control reagent suitable for controlling the functionality of the detection reagent.

In the example illustrated, a feed channel 7 is configured to feed the reference chambers 6 with reference fluid. The feed channel 7 extends in one direction, here the vertical direction, along which the feed chambers are distributed. In this example, the reference chambers 6 are arranged in pairs, one reference chamber 6 on each side of the channel 7, thus forming two columns of reference chambers 6. The reference chambers 6 in a pair are provided with an identical endotoxin concentration. For example, starting from the bottom of FIG. 2, the first pair consists of two reference chambers 6a denoted as “blank”, free of endotoxin, the second pair and the third pair each consist of two reference chambers 6b, each reference chamber 6b of the second pair and the third pair being provided with a first endotoxin concentration of 0.05 EU/mL, EU denoting the endotoxin unit corresponding to an international unit corresponding to 100 pg of endotoxin, the fourth pair and the fifth pair each consist of two reference chambers 6c, each reference chamber 6c of the fourth pair and the fifth pair being provided with a second endotoxin concentration of 0.5 EU/mL, the sixth pair consists of two reference chambers 6d provided with a third endotoxin concentration of 5 EU/mL, the seventh pair consists of two control chambers 6f denoted as “blank”, and free of endotoxin.

The analysis chambers 2 and reference chambers 6 have the same configuration. Typically, these chambers 2, 6 have at least one wall that is transparent to the wavelengths that may be emitted during the reactions, this wall being visible to the imager. The analysis chambers 6 are provided with analytical reagents that are capable of causing a luminescence reaction in the presence of endotoxins in the biological sample with which said reagents are placed in contact. The analytical reagents may be identical to the reference reagents. The reagents comprise, for example, a detection agent in an inactive state in the absence of endotoxin-free activation for the analysis chambers 2 and possibly certain reference chambers 6 (for example control chambers), an agent for activating the detection agent comprising an enzyme and a fluorogenic substrate, and a control reagent suitable for controlling the functionality of the detection reagent.

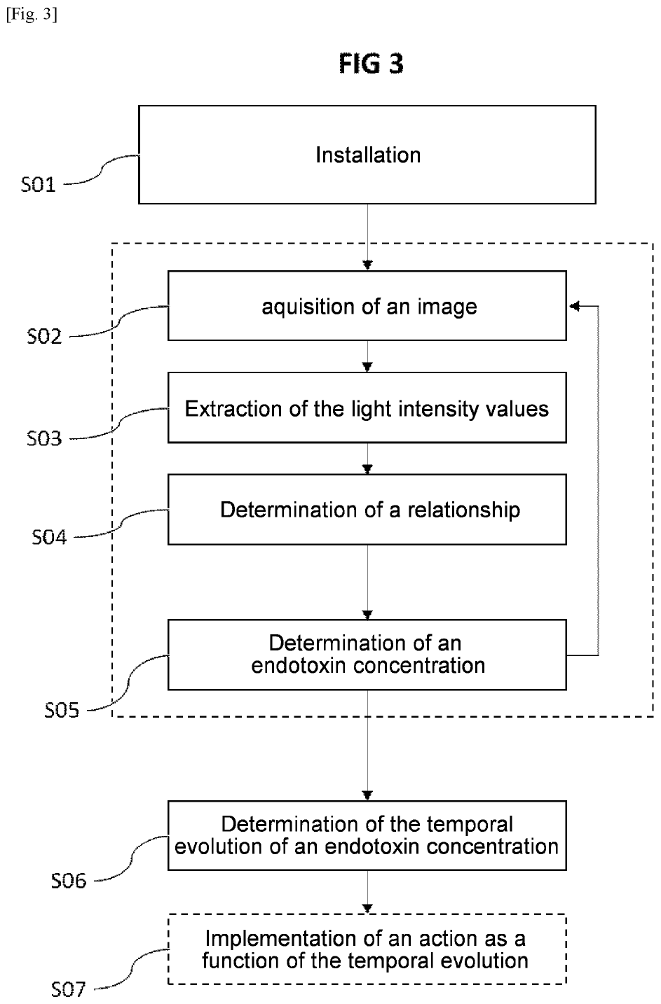

With reference to FIG. 3, in a first installation step S01, the biological sample is placed in the analysis chambers 2, typically by feeding the analysis chambers 2 through the feed channels 4a, 4b, 4c. The reference liquid is also placed in the reference chambers 6 by means of the feed channel 7. The analytical support 1 is then introduced into the analytical instrument 10, in a field of view 11 of the fluorimeter imager 12. Typically, the analytical instrument 10 maintains certain predetermined conditions, such as keeping the biological sample at a given temperature (for example 37° C.).

Next, during the steps that will be described below, several measurement instants of a measurement period are implemented. Obviously, the measuring instrument includes data processing means such as a processor, a memory and an input/output interface, which will not be described in detail. The measurement period typically means the time elapsed between the installation of the analytical support 10 containing the biological sample in the field of view 11 and the last measurement by image acquisition, before measurement is stopped and the results are provided. The measurement period extends over several minutes, usually several tens of minutes, for example over 20 or 40 minutes. The measurement instants are distributed throughout the measurement period, typically with a periodicity of a few minutes, for example every one or two minutes. A measurement period preferably comprises at least five measurement instants, and preferably at least 10 measurement instants. It should be noted that the measurement period may include image acquisitions and measurements which do not form part of the measurement instants for the purposes of the invention, provided that the steps to be described are not performed. In particular, an initial image acquisition may be performed at the start of the measurement period, directed toward determining a reference image intended for processing the other images.

During each measurement instant, an image is acquired (step S02) by the imager 12. In this case, the image is a two-dimensional image made up of spatially organized pixels having coordinates with which are associated light intensity values. As the analytical support 1 is in the field of view 11 of the imager 12, it is an image of the analytical support. Preferably, the analytical support 1 fills the entire acquired image. Moreover, the acquired image may represent the entire analytical support 1, or at least all the chambers 2, 6 of the analytical support 1, or it may represent only some of the chambers 2, 6, in which case it is possible to acquire several images by moving the field of view 11 between two acquisitions relative to the analytical support 1 in order to image all the chambers 2, 6 to be imaged, within a time interval interpreted as a measuring instant. Insofar as acquiring one or more images does not fundamentally change the process, reference will be made hereafter solely to the acquisition of one image, for the sake of non-restrictive simplicity.

Light intensity values of reference chambers 6 and/or analysis chambers 2 are then determined from the acquired image (step S03). In order to improve the results of this determination of the light intensity values, it is possible to perform one or more preprocessing steps on the acquired image.

A first preprocessing step may be the application of one or more corrections to the intensity values of the acquired image pixels, using either correction data predetermined prior to the process and common to all the process implementations, or correction data determined at the start of the measurement period and thus specific to this process implementation. Typically, the correction data take the form of a matrix of image size values, and correction is performed by subtracting or multiplying a pixel light intensity value by a value in the correction matrix.

In particular, correction data may correspond to a dark image, i.e. an image of the field of view 11 acquired in the absence of illumination thereof. The darkness of the field of view 11 means that only the noise caused by dark currents or similar disturbances is seen. The light intensity values of the dark image are subtracted from the intensity values of the acquired image so as to suppress this noise. The correction data may be homogenization data, directed in particular toward correcting any inhomogeneity in the illumination of the analytical support 1 by the analytical instrument 10 or any other, for example optical, homogeneities. These homogenization data may notably take the form of a correction matrix derived from a background image acquired when an object with spatially uniform reflection or fluorescence, for instance an aluminum foil, is present in the field of view 11 and is illuminated. The correction matrix may then contain values allowing any inhomogeneities thus detected to be corrected.

Another correction may be to subtract from the intensity values of the acquired image the corresponding light intensity values of an initial image, acquired at the start of the measurement period, with the analytical support 1 arranged in the field of view 11. Insofar as this initial image corresponds to an instant for which the fluorescence reactions have not yet begun, such a correction highlights in the image acquired from the analytical support 1 only the light variations caused by these fluorescence reactions, and thus dispenses with defects such as dust present on the object generating a spurious fluorescence signal. In other words, this makes it possible to reduce the intensity values to a value relative to the initial value, which is zero in the initial image at the start of the measurement period.

Once any such preprocessing has been performed, the light intensity values of chambers 2, 6 may be extracted from the acquired image (step S03). Preferably, both the light intensity values of the reference chambers 6 and the light intensity values of the analysis chamber(s) 2 are determined at each measurement instant. However, it is possible at a measurement instant to extract only the light intensity values of the analysis chambers 2, for example when it is not necessary to determine the calibration relationship, which will be discussed later, at that measurement instant.

Insofar as the acquired image represents several chambers 2, 6 (for example 35 in the example shown in FIG. 2), it is appropriate to locate each chamber 2, 6 in the acquired image. It is possible to consider that each analysis chamber 2, 6 corresponds to a predetermined location, dictated by the spatial organization of the analytical support 1 and by its known arrangement in the field of view 11. However, such an approach requires precise positioning and fine-tuning of the analytical support 1, and may lead to errors if the actual positioning of the analytical support 1 is not as expected. It is consequently preferable to perform localization of the chambers 2, 6 in the acquired image, for example by means of shape recognition using a shape model (or “template”) corresponding to that of the chambers. An inter-correlation function involving the shape template is applied to the acquired image, determining a similarity score allowing the position of the template in the image, and thus of the chambers 2, 6, to be determined.

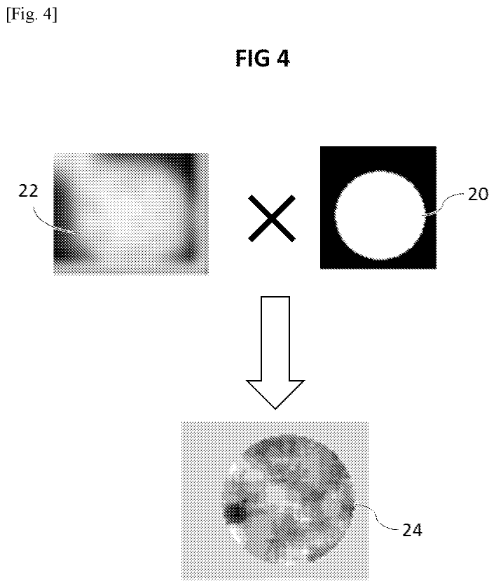

The image may be masked using a mask which is intended to conserve in the image only the areas of the chambers that are to be processed. Typically, the mask has pixels with a value of 1 for the areas of the image to be conserved, and 0 for the rest of the image. In the example shown in FIG. 4, the mask 20 comprises a disk-shaped area to be conserved, in white (value 1), while the rest of the mask 20 is black (value 0). The mask 20 illustrated here covers only a reduced area intended to be centered on an identified location 22 of a chamber 2, 6, which are treated one by one, but the mask 20 might cover several chambers 2, 6. Application of the mask 20 may consist in multiplying the pixel intensity values of each location 22 of a chamber with the corresponding values of the mask 20. This then gives only the area 24 of a chamber 2, 6 which is to be processed, disk-shaped in this example, with pixels outside these areas 24 being zero and not being subsequently taken into account.

An intensity value of a chamber 2, 6 is then derived from the intensity values of the pixels of the chamber thus isolated, so as to be statistically representative thereof, for example by calculating a mean, a median, or a percentile.

Since the support generally comprises several analysis chambers 2, in particular several analysis chambers 2 provided with the same analytical reagents, a statistically representative value of light intensity values of several of said analysis chambers 2 may be determined, from which an analysis chamber light intensity value is derived for the continuation of the process. This statistically representative value may typically be a measure of central tendency such as the mean or, preferably, the median of the light intensity values of said several analysis chambers 2.

It is possible to exclude outlier values, which may correspond to malfunctions, for instance a problem of filling with the biological sample or reference liquid, the presence of an air bubble or dust, etc. It is notably because malfunctions may occur that the analytical support 1 preferably includes several analysis chambers 2, preferably at least three analysis chambers 2, and that each reference endotoxin concentration is associated with several reference chambers 6, preferably at least three reference chambers with the same endotoxin concentration. The exclusion of outlier values may, for example, comprise the comparison relative to a threshold of a deviance criterion for each chamber 2, 6, this preferably being dependent on the values of the intensity values of the other chambers, at least those of the same configuration (analysis chamber 2 or reference chambers 6 with the same endotoxin concentration), for example taking into account a measure of central tendency such as the mean or, preferably, the median. The threshold may, for example, be a deviation relative to the measure of central tendency. Light intensity values exceeding the threshold of a deviance criterion may be discarded and not considered further.

Once the light intensity values of the reference chambers 6 have been extracted, a calibration relationship linking light intensity value and endotoxin concentration is established on the basis of the light intensity values of the reference chambers 6 extracted from the acquired image and previous acquired images. If several reference chambers 6 correspond to the same endotoxin concentration, for instance the control chambers 6a or the four reference chambers 6 with a concentration of 0.05 EU/mL in the example, the intensity values of these reference chambers 6 may be combined, for example via a statistically representative value of these intensity values, for instance a measure of central tendency such as the mean or, preferably, the median.

The calibration relationship takes the form of a function which connects an endotoxin concentration to a light intensity value. In the following example, the calibration relationship links a logarithm of the light intensity values to the logarithm of the endotoxin concentration. Taking logarithms into account allows the calibration relationship to be more error-robust. However, it is possible to use other types of calibration relationship, for instance an affine relationship directly linking the light intensity value to the endotoxin concentration. Other types of regression may be used to determine the calibration relationship, for instance nonlinear regression or even parametric or non-parametric regression. The choice of the type of calibration relationship is made to best reflect the physical relationship between the light intensity value and the endotoxin concentration, and may thus depend on the reagents used and their kinetics.

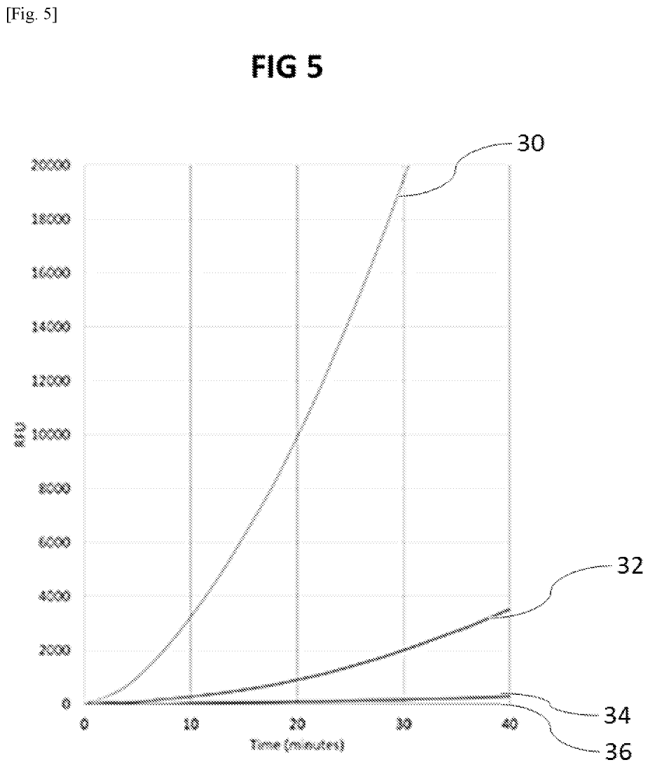

However, the relationship between light intensity value and endotoxin concentration varies over time, due to the kinetics of the reactions involved in the production of fluorescence. FIG. 5 shows examples of the temporal evolution of intensity values in RFU (Relative Fluorescence Unit) for reference chambers 6 associated with endotoxin concentrations of 5 EU/mL (curve 30), 0.5 EU/mL (curve 32), and 0.05 EU/mL (curve 34), and 0 EU/mL (i.e. control chambers 6a, curve 36). The RFU depends on the intensity (amount of photons collected by the imager 12) relative to a reference, with the RFU increasing with intensity. It is possible that the light intensity values of the control chambers 6a are nonzero, and thus reflect fluctuations due to disturbances (which are small and thus not visible in the figure). It is thus possible to subtract the light intensity values of the control chambers 6a from those associated with the different endotoxin concentrations. The other curves 30, 32, 34 clearly show that the temporal evolutions of the light intensity values, while all increasing, show very different kinetics as a function of their respective endotoxin concentrations. A calibration relationship linking light intensity value and endotoxin concentration is thus only valid at the instant of measurement of the data from which this relationship is established.

In order to take these kinetic differences into account, a calibration relationship linking light intensity value and concentration is determined (S04) for each of a plurality of measurement instants, from the light intensity values of the reference chambers 6 at these measurement instants, so as to be able to be used for the data of this measurement instant or subsequent measurement instants. The light intensity values for each reference endotoxin concentration (for example 5 EU/mL, 0.5 EU/mL, 0.05 EU/mL) are thus known at a measurement instant. It is thus possible to establish such a relationship, typically by approximating a function. For example, linear regression may be used, or else interpolation. Preferably, the calibration relationship more precisely links a logarithm of light intensity value and a logarithm of endotoxin concentration. For example, the calibration relationship may link the logarithm of light intensity value and the logarithm of endotoxin concentration into a linear affine function.

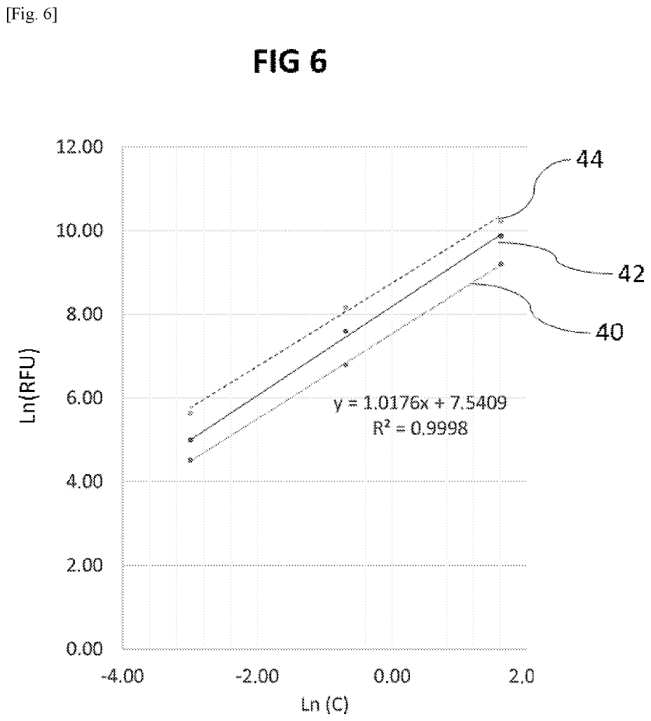

FIG. 6 shows three straight lines 40, 42, 44 illustrating affine functions linking the logarithm of light intensity values (y-axis) to the logarithm of endotoxin concentration C (x-axis). These lines 40, 42, 44 were obtained by linear regression for three measurement instants (20 minutes, 30 minutes, 40 minutes) with known endotoxin concentration values of the reference chambers 6 (C=5 EU/mL, C=0.5 EU/mL, and C=0.05 EU/mL). The first straight line 40 (dotted line) corresponds to the 20-minute relationship, the second straight line 42 (solid line) corresponds to the 30-minute relationship, and the third straight line 44 (dashed line) corresponds to the 40-minute relationship. In this example, the linear regression approximation means that the calibration relationship is of the form ln(Val_{RFT})−a(t)×ln(c)+b(t), with Val_{RFT} the light intensity value, a(t) the directrix at instant t and b (t) a real constant at instant t. For example, the first straight line 40 (at t=20 minutes) has the equation y=1.0176 x+7.5409, for a coefficient of determination of 0.9998. It is clearly seen here that the calibration relationship is not the same depending on the measurement instant. Not only is there an overall shift toward higher light intensity values over time (shift of constant b (t) over time), but also a variation in the directrix a (t) of the lines.

Ideally, a calibration relationship linking the light intensity value and the specific endotoxin concentration is determined at each measurement instant, i.e. each time an image of the analytical support is acquired (step S02). However, it is possible to determine the calibration relationship for only certain measurement instants of the measurement period, and not for other measurement instants of the measurement period. For example, the calibration relationship may be determined only every two or three measurement instants, or with a frequency varying with the progress of the measurement period, and in particular with a higher frequency at the start of the measurement period than at the end of the measurement period. The determination of a calibration relationship may be performed, for example, at least for measurement instants at the start of the measurement period, and then no longer be performed if a stability criterion is met.

For example, such a stability criterion may be sufficient linearity of the calibration relationship (for example between logarithms), typically by comparing a linearization error term with a threshold. Other criteria may be used, notably depending on the type of calibration relationship, for instance limits for correlation coefficients or other parameters.

A specific calibration relationship is determined for at least three measurement instants, preferably for at least five measurement instants, and more preferably for at least eight measurement instants among the instants within the measurement period. A specific calibration relationship is determined at least for measurement instants spread over a period of at least 3 minutes, preferably at least 5 minutes, and more preferably at least 8 minutes. Preferably, a specific calibration relationship is determined for at least a quarter of the measurement instants, and preferably for at least half of the measurement instants.

When a specific calibration relationship is determined at a measurement instant, and in particular when said calibration relationship has met a predefined stability criterion, it is possible to use this same calibration relationship for other subsequent measurement instants. In any case, a calibration relationship is available for each measurement instant, whether specifically determined for that measurement instant or inherited from a determination in connection with a preceding measurement instant. In addition, not all the measurement instants within the measurement period have the same calibration relationship.

For each measurement instant, the calibration relationship allows at least one measurement of endotoxin concentration in the biological sample to be determined from the intensity value associated with the analysis chambers at said measurement instant (step S05). Specifically, the calibration relationship links the light intensity value and the endotoxin concentration, so that by measuring a light intensity value, a corresponding endotoxin concentration measurement is found for that instant. For example, taking a calibration relationship of the form ln(Val_{RFT})−a(t)×ln(c)+b(t), this may be written:

$$ln ( C ) = \frac{ln ( {Val}_{RFU} ) - b ( t )}{a ( t )}$$

and the endotoxin concentration c of the sample sought at instant t is thus

$$C ( t ) = {e}^{\frac{ln ( {Val}_{RFU} ) - b ( t )}{a ( t )}}$$

Needless to say, the expression of the endotoxin concentration as a function of the light intensity value depends directly on the expression of the calibration relationship, and may thus take an entirely different form.

As this determination of the endotoxin concentration in the sample is performed for several measurement instants during the measurement period, the endotoxin concentration is precisely known at each of these measurement instants.

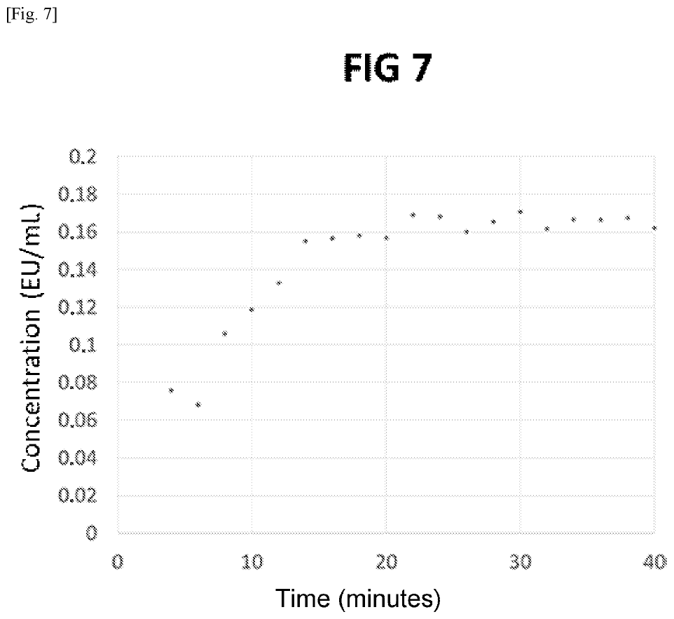

It is thus possible to determine the temporal evolution of an endotoxin concentration measurement in the biological sample over the measurement period (step S06). FIG. 7 shows an example of temporal evolution of the endotoxin concentration measurement in the sample (in Eu/mL), as a function of time (in minutes), obtained by using, for each measurement instant (represented by dots), a calibration relationship specific to that measurement instant. It is notably possible to observe stabilization of the endotoxin concentration after 15 minutes. More specifically, the first few minutes correspond to a transitory regime in which resuspension and homogenization of the reagents in the liquid sample present in the analysis chambers 2 take place, while thereafter the enzyme-substrate reactions become organized, resulting in a stabilized regime of these reactions, reflected by the observed stabilization of the measured endotoxin concentration value.

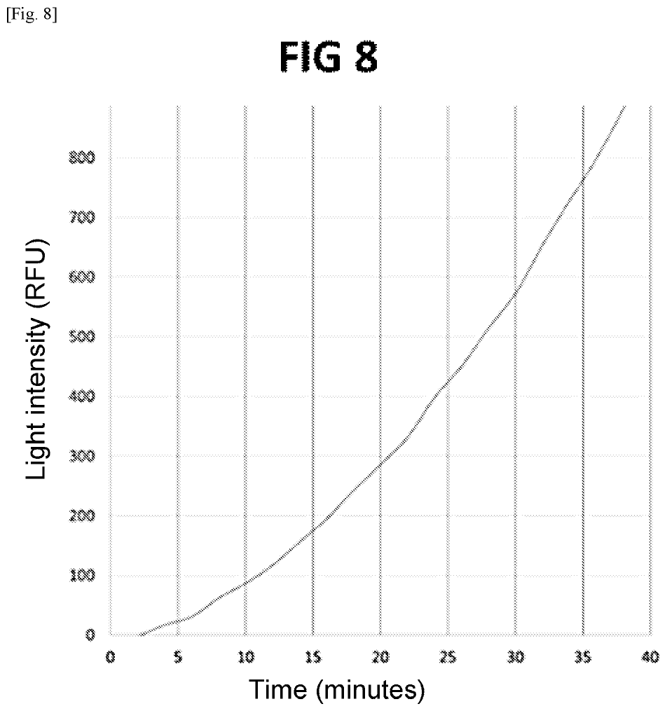

By way of comparison, FIG. 8 shows the temporal evolution of light intensity values for the example in FIG. 7. More precisely, the curve in FIG. 8 illustrates the temporal evolution, over the measurement period, of the logarithm of the mean light intensity values of seven analysis chambers 2 receiving the biological sample. In contrast to the temporal evolution of the endotoxin concentration measurement in FIG. 7, the light intensity values show a continuous increase over time. However, the fluorescence intensity is not directly representative of the endotoxin concentration. Rather, it is the respective kinetics of light intensity increase that is linked to the endotoxin concentration. Stabilization of the measured endotoxin concentration value is reflected by the stabilization of the slope of the curve in FIG. 8, relative to the calibration relationship.

The light intensity values alone thus do not reflect the stabilization found after 15 minutes. Only the proposed process, with a calibration relationship that evolves with the measurement instants, and thus with time, is able to account for this stabilization.

The determination of the temporal evolution of the endotoxin concentration measurement may be made only at the end of the measurement period, from the mass of measurement instants. However, this determination of the temporal evolution of the endotoxin concentration measurement in the sample may be performed as soon as the light intensity values of the reference and analysis chambers are available for at least two measurement instants. Consequently, the determination of the temporal evolution of the endotoxin concentration measurement is performed several times during the measurement period, following several measurement instants. A kind of temporal evolution update of the endotoxin concentration measurement is thus achieved. Determination of the temporal evolution may notably be performed after each measurement instant, once the light intensity values of the reference and analysis chambers are available for at least two measurement instants, or once a given number of measurement instants has been reached. This determination may also be periodic, with a period greater than the interval between two measurement instants.

Knowledge of the temporal evolution of the endotoxin concentration measurement in the sample has several advantages. For example, it allows the detection of a possible malfunction if this temporal evolution shows anomalies, for instance a pronounced decrease, which might not be detected with a single isolated measurement at the end of the measurement period. Updating the temporal evolution of the endotoxin concentration measurement moreover allows such anomalies to be detected before the end of the measurement period initially planned, and thus allows the sample analysis to be stopped, for example, in order to be restarted, thus affording considerable time savings.

As in the example shown in FIG. 7, it is also possible to detect a stabilization of the endotoxin concentration (for example with variations below a given threshold) over several measurement instants (for example over three to six measurement instants, or over a period of at least 3 minutes, preferably at least 5 minutes), and then to stop the sample analysis. In the example shown in FIG. 7, stopping the measurement after 20 minutes would have allowed the measurement period to be halved, thereby proportionately speeding up the quantification of the endotoxins in the biological sample.

It is thus possible to perform an action as a function of the temporal evolution of the endotoxin concentration measurement (step S07), for instance in the event of anomaly or stabilization of the concentration, stopping the measurement and/or triggering an alert to the operator (visual or audible indication, for example), or any other action making it possible to exploit knowledge of the temporal evolution of the endotoxin concentration measurement in the sample.

As mentioned previously, stabilization of the temporal evolution of the endotoxin concentration measurement, at a sufficiently high level, may show that the expected reactions have taken place in full, and that an extension of the measurement period is not necessary. Stopping the measurement is thus triggered as a function of the temporal evolution of the endotoxin concentration measurement, and not as a function of a predetermined time. Typically, the conditions for stopping the measurement on this stability criterion include endotoxin concentration values showing variations therebetween below a variation threshold, for instance over the last three to six measurement instants, or over measurement instants of a predefined duration, for instance a duration of at least 3 minutes, preferably at least 5 minutes. This approach makes it possible to minimize the time required to obtain measurement results, as a function of an acceptable quantification deviation.

In the absence of stabilization of the temporal evolution of the endotoxin concentration measurement, the measurement continues with new measurement instants until this stability is reached or a time limit is reached. In the absence of this stability after a certain time, the measurement can be stopped and/or an alert can be issued. A malfunction can thus be detected more quickly.

The validity of the endotoxin quantification may be affected by the absence of stability in the temporal evolution of the endotoxin concentration measurement, and notably the results may be invalidated due to this absence. This enables the user to avoid taking into account final endotoxin quantification results which may turn out to be erroneous due to a malfunction highlighted by the absence of stability.

This is likewise the case for a pronounced decrease in the endotoxin concentration measurement, revealing a malfunction. Even in a stabilized regime such as that shown in FIG. 7, the endotoxin concentration measurement shows slight fluctuations, which are normal. On the other hand, it would be abnormal to find a sharp decrease in this endotoxin concentration measurement. It is thus possible to use a maximum decrease threshold to detect such abnormal behavior, the crossing of which may cause the process to be stopped or an alarm to be issued as a function of a decrease in the endotoxin concentration measurement.

The temporal evolution of the endotoxin concentration measurement may also be used to demonstrate the absence of expected reactions, for example in the absence of endotoxin in the biological sample, by verifying that the endotoxin concentration measurement has not risen transiently during the measurement period.

Needless to say, a minimum threshold for the endotoxin concentration values, allowing reactions to be ensured, can be provided for the implementation of some of the abovementioned actions.

At the end of the measurement period, whether this is due to the stability of the endotoxin concentration measurement, the expiry of an allotted time, or the detection of an anomaly, it is then possible to provide analysis results derived from the temporal evolution of the endotoxin concentration, before the process is stopped, or at the time the process is stopped. It should be noted that the action which is a function of the temporal evolution may also be the continuation of the measurement if the temporal evolution does not present a reason for stopping or issuing an alert.

The invention is not limited to the embodiment described and represented in the attached figures. Modifications remain possible, notably in terms of the way in which the various technical features are constituted, or by substituting technical equivalents, without, however, departing from the scope of protection of the invention.

1. A process for quantifying endotoxins in a biological sample via an analytical instrument comprising an imager defining a field of view, an analytical support being introduced into the field of view of the analytical instrument, said analytical support comprising at least one analysis chamber configured to receive said biological sample and a plurality of reference chambers configured to receive a reference liquid, the process first involving placing the biological sample in said analysis chamber and reference liquid in the plurality of reference chambers, reference chambers being provided with reference reagents and different concentrations of endotoxins, said reference reagents being capable of causing a luminescence reaction in the presence of the reference liquid as a function of the endotoxin concentration in the corresponding reference chamber, the analysis chamber being provided with analytical reagents that are capable of causing a luminescence reaction in the presence of endotoxins of the biological sample,
the process comprising, for each measurement instant of a measurement period, the acquisition at said measurement instant of an image of the analytical support and the determination, from said image of the analytical support, of an analysis chamber light intensity value for said measurement instant,
the process also comprises, for a plurality of measurement instants:
the determination; from the image of the analytical support, of light intensity values of reference chambers for said measurement instant,
the determination, from the light intensity values of reference chambers for said measurement instant, of a calibration relationship linking the light intensity value and the endotoxin concentration at said measurement instant;
the process also comprising, for a plurality of measurement instants:
the determination of at least one measurement of endotoxin concentration in the biological sample at said measurement instant from a calibration relationship for said measurement instant and the analysis chamber intensity value at said measurement instant; and
the determination of a temporal evolution of the endotoxin concentration measurement in the biological sample over the measurement period from endotoxin concentration measurements for several measurement instants.

2. The process as claimed in claim 1, subsequently comprising the implementation of an action as a function of the temporal evolution of the endotoxin concentration measurement.

3. The process as claimed in claim 2, in which the action performed involves stopping the process or issuing an alert as a function of the stability of said temporal evolution of the endotoxin concentration measurement or of a decrease in the endotoxin concentration measurement.

4. The process as claimed in claim 1, in which a calibration relationship is determined for each measurement instant of the measurement period.

5. The process as claimed in claim 1, in which the calibration relationship for a measurement instant is a calibration relationship determined from the light intensity values of the reference chambers of a preceding measurement instant.

6. The process as claimed in claim 1, in which the temporal evolution of a measurement of endotoxin concentration in the biological sample is determined several times during the measurement period, each time following a measurement instant taken into account in said temporal evolution.

7. The process as claimed in claim 1, analytical reagents present in analysis chambers and reference reagents present in reference chambers comprising a recombinant factor C and a fluorogenic substrate of the reference chambers comprising endotoxins at predetermined concentrations.

8. The process as claimed in claim 7, in which at least one reference chamber is free of endotoxin.

9. The process as claimed in claim 1, in which the analytical support comprises a plurality of analysis chambers, and the determination of an analysis chamber light intensity value for said measurement instant comprises the determination of a statistically representative value of light intensity values of a plurality of said analysis chambers.

10. An analytical instrument comprising an imager defining a field of view, the analytical instrument being configured to receive an analytical support in the field of view of the imager, said analytical support comprising at least one analysis chamber configured to receive a biological sample, and a plurality of reference chambers configured to receive a reference liquid, the reference chambers being provided with reference reagents and reference concentrations, the reference reagents being capable of causing a luminescence reaction in the presence of the reference liquid as a function of the endotoxin concentration in the corresponding reference chamber, the analysis chamber being provided with analytical reagents that are capable of causing a luminescence reaction in the presence of endotoxins of the biological sample, the system being configured to perform at least the steps of the process as claimed in claim 1.
